(19) United States
(12) Patent Application Publication
(10) Pub. No.: US 2004/0220529 A1
(43) Pub. Date: Nov. 4, 2004

(54) Apparatus and methods for activating a delivery device

(76) Inventors: James E. Brown, Los Gatos, CA (US); Edward M. Gillis, San Jose, CA (US); Andrew I. Poutiatine, Redwood City, CA (US); William D. Kolosi, San Francisco, CA (US); James R. Flom, San Francisco, CA (US); Felix Theeuwes, Los Altos Hills, CA (US)

Correspondence Address: DURECT CORPORATION, 10240 BUBB ROAD, CUPERTINO, CA, 95014, US

(21) Appl. No.: 10/687,054

(22) Filed: Oct. 15, 2003

Related U.S. Application Data
(60) Provisional application No. 60/419,273, filed on Oct. 16, 2002.

(51) Int. Cl. A61M 5/00

(52) U.S. Cl. 604/208

(57) ABSTRACT

Devices and methods of actuating/activating agent delivery systems are provided. The subject devices include a trocar into which an agent delivery device is loaded in preparation for implantation of the agent delivery device into a patient. The action of loading the agent delivery device into the trocar serves to actuate or activate the device for the delivery of the agent which will commence upon implantation of the device. The methods include use of the trocar to actuate/activate the agent delivery device.

This disclosure claims the benefit of pending U.S. provisional application No. 60/419,273, filed Oct. 16, 2002.

### FIELD OF THE INVENTION

The present invention generally relates to devices for activating or actuating an action or function of a device, and is particularly useful for ensuring the timely activation or actuation of the controlled delivery of an agent from an implantable delivery device.

### BACKGROUND OF THE INVENTION

Controlled delivery of beneficial agents such as drugs in the medical and veterinary fields has been accomplished by a variety of methods that may employ various types of drug delivery devices. A range of exemplary devices and methods are well described in the “Encyclopedia of Controlled Drug Delivery” 1999, published by John Wiley & Sons Inc, edited by Edith Mathiowitz. Drug delivery devices, including implantable devices, can be based on, for example, diffusive, erodible or convective systems, e.g., pumps, such as osmotic pumps, that may or may not be connected to a catheter, biodegradable implants, electrodiffusion systems, electroosmosis systems, vapor pressure pumps, electrolytic pumps, effervescent pumps, piezoelectric pumps, electrochemical pumps, erosion-based systems, electromechanical systems, liposomes, depots, or microspheres. Every containerized device requires an orifice of one sort or another, and such an orifice must address the particular needs of drug delivery in a certain situation, such as the need for low flow rate, steady, predictable flow rate and the need to keep the orifice closed until flow is required.

One approach for delivering a beneficial agent involves the use of implantable diffusional systems. For example, subdermal implants for contraception are described by Philip D. Damey in Current Opinion in Obstetrics and Gynecology, 1991, 3:470-476, Norplant®. Six levonorgestrel-filled silastic capsules are placed under the skin to provide protection from conception for up to 5 years is achieved. The implants operate by simple diffusion, that is, the active agent diffuses through the polymeric material at a rate that is controlled by the characteristics of the active agent formulation and the polymeric material.

Another method for controlled prolonged delivery of a beneficial agent involves the use of an implantable osmotic delivery system. Osmotic delivery systems are very reliable in delivering the beneficial agent over an extended period of time. The osmotic pressure generated by an osmotic pump also produces a delivery rate of the beneficial agent into the body which is relatively constant as compared with other types of delivery systems.

In general, osmotic delivery systems operate by imbibing fluid from the outside environment and releasing corresponding amounts of the beneficial agent. Osmotic delivery systems, commonly referred to as “osmotic pumps,” generally include some type of a capsule having walls which selectively pass water into an interior of the capsule which contains a water-attracting agent. The absorption of water by the water-attracting agent within the capsule reservoir creates an osmotic pressure within the capsule which causes the beneficial agent to be delivered from the capsule. The water-attracting agent may be the beneficial agent delivered to the patient, however, in most cases, a separate agent is used specifically for its ability to draw water into the capsule.

When a separate osmotic agent is used, the osmotic agent may be separated from the beneficial agent within the capsule by a movable dividing member or piston. The structure of the capsule is such that the capsule does not expand when the osmotic agent takes in water. As the osmotic agent expands, it causes the movable dividing member or piston to move, which in turn causes the beneficial agent to be discharged through an orifice at the same volumetric rate that water enters the osmotic agent by osmosis.

Another method for controlled prolonged delivery of a beneficial agent involves the use of an implantable chemical or electrochemical delivery system. A controlled delivery device for holding and administering a biologically active agent includes a housing which encloses a displacing member, a chemical or electrochemical cell that generates pressure, and may include activation and control circuitry. The electrochemical or chemical cell generates gas within the housing, forcing the displacing member against the beneficial agents contained within the housing and forcing the beneficial agents through an outlet port and into the environment of use at a predetermined rate.

The orifice in any of the above devices controls the interaction of the beneficial agent with the external fluid environment. The orifice serves the important function of isolating the beneficial agent from the external fluid environment, since any contamination of the beneficial agent by external fluids may adversely affect the utility of the beneficial agent. For example, the inward flux of materials of the external fluid environment due to diffusion or osmosis may contaminate the interior of the capsule, destabilizing, diluting, or otherwise altering the beneficial agent formulation. Another important function of the orifice is to control or limit diffusional flow of the beneficial agent through the orifice into the external fluid environment.

In known delivery devices, these functions have typically been performed by flow moderators. A flow moderator may consist of a tubular passage having a particular cross sectional area and length. The cross sectional area and length of the flow moderator is chosen such that the average linear velocity of the exiting beneficial agent is higher than that of the linear inward flux of materials in the external environment due to diffusion or osmosis, thereby attenuating or moderating back diffusion and its deleterious effects of contaminating the interior of the osmotic or diffusion pump. In addition, the dimensions of the flow moderator may be chosen such that the diffusive flux of the beneficial agent out of the orifice is small in comparison to the convective flux.

One problem with flow moderators, however, is that the passage may become clogged or obstructed with particles suspended in the beneficial agent or in fluid from the external environment. Such clogging may be reduced or eliminated by increasing the diameter of the passage to 5 mils or more, for example. However, this increase results in a greater rate of diffusion of the beneficial agent out of the pump. A corresponding increase also occurs in the back diffusion of the external fluid into the pump which may contaminate the beneficial agent and adversely affect the desired delivery rate of the beneficial agent. Tolerances during fabrication also frequently dictate that the orifice diameter be greater than about 5 mils.

Systems with a long straight flow moderator are also impractical for implantation applications because they increase the size of the implant significantly making the system difficult to implant.

Leakage of the beneficial agent from the pump or device, prior to implanting the same, may occur due to pressure changes in the reservoir containing the beneficial agent caused by changes in temperature of the environment that the pump is being stored in. Loss of beneficial agent to the environment through evaporation is another common occurrence to varying degrees during the storage or shelf life of various implantable pumps.

Another problem associated with pressure driven implantable drug delivery devices is know as the burst effect, wherein, due to thermal expansion of a drug or other beneficial agent upon removing the implantable device from a room temperature, shelf environment and implanting it into an environment at body temperature, an initial volume or bolus of the drug or beneficial agent is delivered from the device which is often much larger than a predetermined measured dose called for. This phenomenon can be a critical problem, causing severe damage or even death to the patient in the worst scenarios.

Current flow modulators also cause separation of beneficial agents which contain suspensions of bioactive macromolecules (proteins, genes, etc.). When such suspensions pass along a restriction in current flow modulators, the suspension separates and the delivery concentration of bioactive macromolecules varies.

Additionally, if a drug formulation is allowed to sit in a delivery outlet channel during storage, then precipitation of solutes out of solution (due to evaporation and surface effects) may cause the delivery outlet channel to become blocked with precipitated solute.

The above problems are particularly acute when the drug to be delivered is highly potent, when the volume to be delivered is small, and when delivery is done over a prolonged period of time.

The assignee of the present invention has invented implantable pumping devices having a positively actuated (i.e., pushed, pulled, or turned) valve on the exit orifice of the reservoir of the devices which addresses the drawbacks of prior devices. Specifically, the valve allows for the system to be closed during its shelf life and thus prevents the exit of fluid out of the reservoir of the system during storage. Prior to operation of the system, the valve is positively actuated or opened by the user. The configuration of the valve allows room for thermal expansion of the contents of the delivery device or pump after it has been actuated. This controls the release of drug substance which is contained in the pump due to the increase of the temperature of the pump contents from room temperature to body temperature. When open, the valve allows for a fully patent outflow track or exit orifice for the agent to exit the delivery device thereby providing an even flow of the contents from the pump during operation. Additionally, the valve is designed to accommodate pressure buildup which occurs in the reservoir of the closed system during temperature and atmospheric pressure cycling of the system in storage. Thus, the valve solves the problems of keeping the system closed until needed, controls drug burst due to differential thermal expansion of the drug and the container, reduces precipitation of drug causing blockage of the outflow channel, and provides a uniform, even and predictable flow of drug out of the drug delivery device. Various embodiments of such devices and their respective valves are disclosed in PCT Patent (published) Application No. WO/0054745, filed Mar. 16^{th}, 2000, and published 21 Sep. 2000, and U.S. in patent application Ser. No. 10/188,325 filed on Jun. 27^{th}, 2002, which are herein incorporated by reference.

As mentioned, such devices may be activated or actuated to be in an open condition prior to or upon implantation of the delivery device. The devices may be configured to be activated by manually contacting an exposed end of the valve or by grasping an extension thereof to push, pull or rotate the valve. Alternatively, the valves may be configured (e.g., may have a Phillips head slot, and Allen receptacle, or the like) to receive a tool manipulated by the user or physician.

Because a positive actuation or an active step on the part of the user implanting the device is required, there are certain risks associated with such valved delivery devices. The user might unintentionally open the valve prematurely, e.g., prior to proper implant of the device, risking contamination within the device or leakage of the agent contained within the device. Even if the valve is properly implanted prior to activating or opening the valve, the area or part of the valve which is to be engaged, pushed, pulled or rotated for purposes of activation may now be difficult to access. Additionally, and more importantly, human error may lead to incomplete or incorrect activation of the device or the user may forget to activate the device altogether.

Thus, there is a need for devices and methods which eliminate the handling steps and the risk of human error involved in the positive or active actuation of certain devices such as implantable agent delivery devices. Preferably, such devices and methods would obviate the need for user activation of such a device by incorporating such activation with the act of delivering or implanting the device to an intended location, such as a location within the body.

### SUMMARY OF THE INVENTION

Devices and methods for actuating a function of another device, such as an implantable drug delivery device, are provided. In one embodiment, the subject apparatus includes an elongated tubular member having a lumen extending from a proximal end to a distal end, wherein the lumen is adapted to translatably receive the device therein. The apparatus further includes a retaining mechanism located within the lumen at the distal end for retaining the device within the lumen when the device is translated proximally within the lumen. A resistive load means is positioned within the lumen and adapted to contact the inserted device received within the lumen, where the resistive load means has a first resistive load level and a second resistive load level wherein the second resistive load level is greater than the first resistive load level. A rod is also positioned within the lumen proximal to the resistive load means and translatable within the lumen. A locking mechanism is provided within the lumen for locking onto the rod. As a force is applied to the device which in turn applies the force to the resistive load means wherein, when the applied force is sufficient to overcome the first resistive load level, the device function is activated and, when the applied force is sufficient to overcome the second resistive load level, the locking mechanism locks onto said rod.

The resistive load means may be any means that applies resistance to a force, such as a spring, or any other means having elastic properties.

The resistive load level may be, for example, from about 0.1 newtons to 15 newtons, or from about 0.5 N to 10 N or from about 1 N to 5 N.

An alternative way in which an implantable drug delivery device, or other device, may be actuated, involves electrically or electromagnetically, opening or closing a channel. The actuation and implantation device of the invention when in use, may cause a valve to be opened or closed. For example, a solenoid or inductive coil may be employed, whereupon actuation a current is passed through the solenoid producing electromagnetic effect resulting in the opening or closing of the valve. The force applied to the device to counteract the resistive load may be used to concomitantly energize the solenoid and thereby open the valve. Such energization may be done by physically contacting the circuitry of a solenoid (or other inductive mechanism) to a circuit or energy source within the body of the actuation and implantation device. Alternatively, energization may be achieved by wireless transmission of en electromagnetic signal from the actuation and implantation device to the device to be actuated.

Another alternative way in which a device may be actuated involves the activation of an implanted device that includes an electrochemical pump. In such an embodiment an electric circuit in the implanted device may be closed (or opened) upon implantation by methods described above. This can result in a flow of current through an electrochemical device such that an internal pumping effect is achieved and a fluid contained within the device is delivered. Such electrochemical pump devices are disclosed, for example, in U.S. application Ser. Nos. 6,491,684, and 6,575,961, both incorporated by reference herein.

These and other features of the invention will become apparent to those persons skilled in the art upon reading the detailed description below.

### BRIEF DESCRIPTION OF THE DRAWINGS

The present invention will be more readily understood upon reading the following detailed description in conjunction with the drawings, wherein like reference numerals are used throughout the drawings to designate like components.

FIG. 1A illustrates an agent delivery device usable with the activating/actuation device of the present invention where the agent delivery activation mechanism, e.g., plug or valve, is in a closed position.

FIG. 1B is enlarged view of the valve mechanism of the agent delivery device of FIG. 1A wherein the valve mechanism is in a closed condition.

FIG. 1C is enlarged view of the valve mechanism of the agent delivery device of FIG. 1A wherein the valve mechanism is in an open condition.

FIG. 2 is a cross-sectional side view of one embodiment of an actuation/activation device of the present invention.

FIG. 3 is a cross-sectional top view of another embodiment of an actuation/activation device of the present invention.

FIG. 4 illustrates one possible engagement or loading arrangement of an implant device and an actuation/activation device of the present invention.

FIG. 5 illustrates another possible engagement or loading arrangement of an implant device and an actuation/activation device of the present invention.

FIG. 6 illustrates another possible engagement or loading arrangement of an implant device and an actuation/activation device of the present invention.

### DETAILED DESCRIPTION OF PREFERRED EMBODIMENTS

Before the actuation/activation devices and methods of the present invention are described, it is to be understood that this invention is not limited to particular mechanisms described, as such may, of course, vary. It is also to be understood that, while only particular embodiments of implantable delivery devices are referenced or described herein for use with the actuating/activating devices of the present invention, other similar delivery devices or any device requiring activation or actuation where there is a significant risk of user error may be actuated with the subject actuating/activating devices. As such, reference to specific embodiments of implantable delivery devices is solely for the purpose of describing the subject invention and is not in any way intended to limit the scope or function of the subject invention. Moreover, it is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a ring” or “an O-ring” includes a plurality of such rings or O-rings and reference to “the flow path” includes reference to one or more flow paths and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “agent” includes water, an electrolyte, any physiologically or pharmacologically active substance or substances, or combinations thereof. An agent may further optionally include other pharmaceutically acceptable carriers and/or additional ingredients such as antioxidants, stabilizing agents, permeation enhancers, etc.

The terms “activation” and “actuation” and their respective derivations are herein used interchangeably.

The term “implant” includes any delivery devices which requires activation/actuation of a function. These implants include but are not limited to devices that deliver an agent such as a drug, where activation may allow delivery of the drug from the device, and also may include electrical devices, where activation may energize the device.

In the following description, the present invention will be described in the context of implantable devices for the delivery of beneficial agents, however, the present invention may be employed with any device requiring activation or actuation of a certain action or function to be performed by the device. An exemplary agent delivery device will be described first followed by a description of the subject actuating/activating devices and the methods of actuating/activating the delivery of agent from the delivery devices. Finally, a review is provided of the kits of the present invention which include the subject actuating/activating devices.

Exemplary Agent Delivery Device

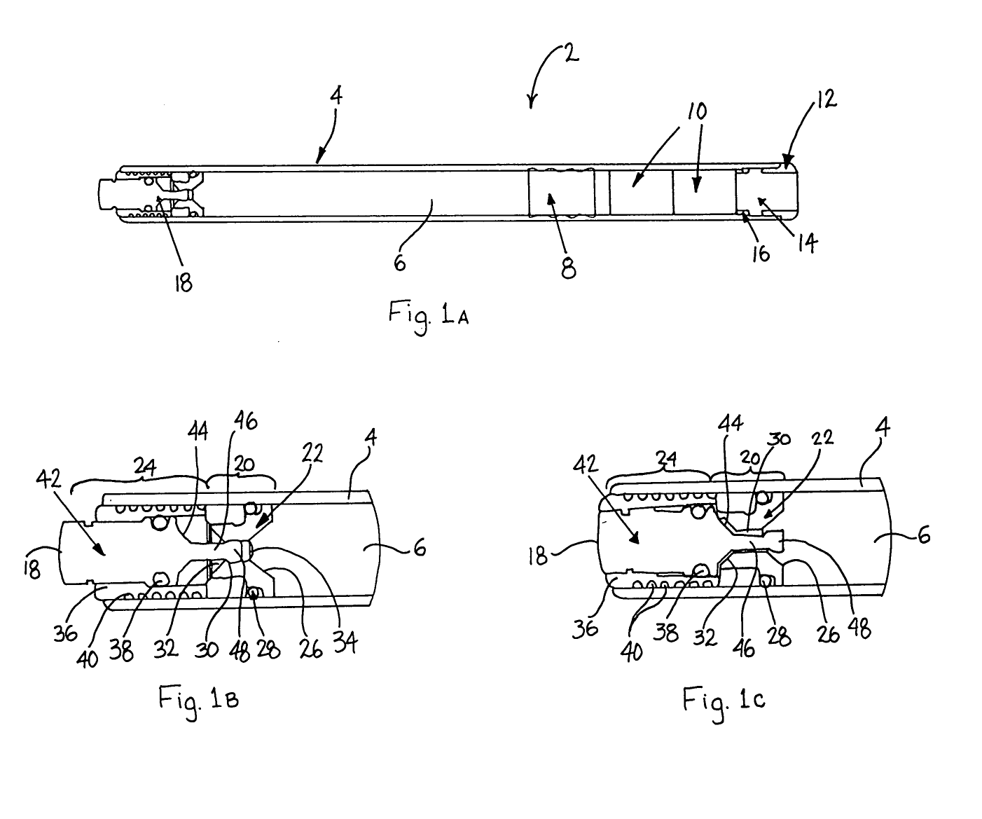

An example of an implantable agent (or drug) delivery device usable with the present invention, and disclosed in PCT Patent Application No. WO/0054745, is illustrated in FIGS. 1A and 1B. While reference can be made to the above-referenced document for a detailed description of anastomotic connectors usable with the present invention, a brief description is herein provided for purposes of convenience.

Drug delivery device 2 includes a housing or pump body 4 that houses a reservoir 6 for holding a drug or beneficial agent to be delivered from device 2. Housing 4 further contains a piston or other driver 8, which separates the drug reservoir 6 from another chamber or chambers 10 that house a piston driving means, such as osmotic tablets. On the other side of chambers 10 is a cap 12 which contains a membrane 14 therein through which the osmotic reaction takes place. A retaining ring 16 maintains cap 12 in sealing engagement with membrane 14.

At the opposite end of agent delivery device 2 is located a bottom assembly 20 and a top assembly 24 which together form a valving mechanism. Top assembly 24 is secured in the open end of housing 4 with a bottom end of the top assembly abutting the top end of bottom assembly 20. Top assembly 24 includes a plunger 18 fitted within a cylindrical ring 36 and at least one O-ring 38 forming a seal there between. About the perimeter of cylindrical ring 36 is defined a helical channel 40 through which the drug/beneficial agent travels when released for delivery. Plunger 18 has a body portion 42, which tapers at a shoulder 44, and a neck portion 46 which extends from shoulder 44. Neck portion 46 terminates in head portion 48 which has a diameter dimension sufficient to provide a sealing engagement with a valve neck 30 of bottom assembly 30.

Bottom assembly 20 is secured within housing 4 adjacent reservoir 6 with a bottom end 26 of the bottom assembly contacting the drug/beneficial agent contained within reservoir 6. Bottom assembly 20 includes a valve seat member 22 and at least one O-ring 28 forming a seal between the valve seat member 22 and inner walls of housing 4. O-ring 28 assures that leakage/evaporation of the drug/beneficial agent does not occur between the bottom assembly 20 and housing 4. Valve seat member 22 has a top cone 32 and a bottom cone 26 bridged by a valve neck 30. Bottom cone 26 acts to focus or funnel the drug/beneficial agent into the valve neck 30 while top cone 32 acts to focus the drug/beneficial agent from valve neck 30 into channel 40 of cylindrical ring 36. Valve seat member 22 is positioned such that a meniscus 34 is formed by the top of the drug/beneficial agent.

As depicted in FIG. 1B, during storage of and prior to implantation of device 2, plug 18 is in a partially extended position (i.e., extending distally from housing 4) within housing 4 to maintain a closed system reservoir 6. When it comes time to implant device 2 or otherwise place it in an environment of use, the valving mechanism is actuated to an open position as shown in FIG. 1C, thereby opening reservoir 6 to flow path 40 that leads out of device 2 for delivery of the drug/beneficial agent to the environment of use. To open the valve mechanism, plunger 18 is depressed relative to housing 4 to a position where the top of plunger 18 is substantially flush with the top end of cylindrical ring 36. Once plunger 18 is depressed, top O-ring 38 maintains plunger 18 in the depressed position. This actuation forces plunger head 48 out of valve neck 30 and into the volume defined by bottom cone 26, thereby breaking the seal formed between plunger head portion 48 and the valve neck 30. This action provides a flow from reservoir 6 through valve neck 30 and between top cone 32 and shoulder 44 of plunger 18. This flow path in turn flows into channel 40 of cylindrical ring 36 and bordered by the inner walls of housing 4. The flow pathway thus defined forms a widely patent or open exit channel which allows the uniform flow of drug/beneficial agent from the reservoir 6 to the environment of use during the operation of the pumping system as it provides a driving force to the reservoir 6.

While the subject invention is especially useful for actuating or activating the agent delivery device just described, it will be obvious to those skilled in the art that the subject invention may be employed with variations of such agent delivery devices. As such, reference to specific embodiments of agent delivery devices is solely for purposes of describing the subject invention and is not in any way intended to limit the scope or the function of the subject invention.

Actuation/Activation Devices and use Thereof

In a preferred embodiment of the subject actuation/activation devices, the devices take the form of a trocar or an elongated tubular member into or on which the device to be activated or actuated is inserted or mounted. In the context of implantable devices (also referred to herein as “implants”), the trocar may also act as a tool for implanting the implantable device or otherwise provide a means by which to deliver the implant to within a selected location within the body. By activation or actuation of the implant it is meant that the delivery of an agent operatively contained within the implant is activated or actuated, i.e., the controlled delivery from the implant is commenced or made possible to commence.

The trocar may be configured such that it activates or actuates, e.g., by applying a load or force to, the implant either upon or integral with loading the implant into or onto the trocar prior to implantation of the implant. For example, the trocar may be configured such that improper activation of the implant may prevent engagement of the implant into the trocar or disengagement of the implant from the trocar. As such, integrating the activation/actuation of the implant with the loading/unloading of the implant greatly minimizes the risk of user-related errors such as the improper activation of the device or forgetting to activate the implant. To this end, the trocar may employ various types of activation or actuating mechanisms. For example, the actuating mechanism could be configured to open an orifice within the implant, to depress a button, to actuate an electronic switch or to break a septum or seal on the implant.

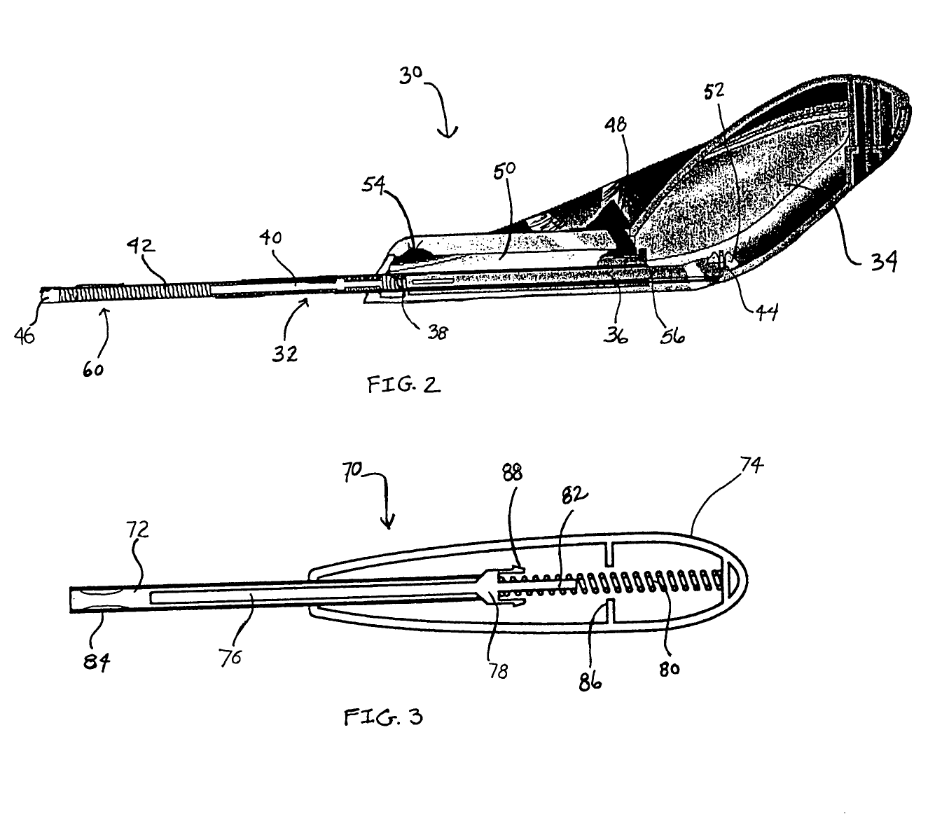

FIG. 2 illustrates one embodiment of an actuation/activation device 30 of the present invention. Device 30 includes a trocar or elongated tube 32 and a handle 34 attached at a proximal end 44 of trocar 32. Trocar 32 defines a lumen therein and preferably has a pointed and sharp distal end 46 to facilitate penetration into the patient, such as into the sub-dermal tissue. Coaxially aligned within the lumen of trocar 32, from open distal 46 to proximal end 44, are a first load member 42, a load spacer 40, a second load member 38 and a push rod 36. The push rod holds the implant in place while the user retracts the trocar. Second load member 38 provides a resistive load which is greater than first load member, 42, typically about 2 to about 10 about times greater where the first load member has a resistive load in the range from about 0.5 to about 10 lbf. In the embodiment illustrated in FIG. 2, the load members are in the form of springs where first load member 42 is a light spring having a resistive load of about 1 lbf and second load member 38 is a heavy spring having a resistive load of about 5 lbf.

The use of two load members, the respective purposes of which are discussed below, is only exemplary. The trocar of the present invention may employ one load member as illustrated in FIG. 3 (where there is no need for a load spacer) or three or more load members to carry out the function of two load members (where additional load spacers may be required). Regardless of the number of load members employed, at least two resistive load levels are provided thereby wherein the second load level is greater than the first load level. Additionally, while the illustrated embodiments employ springs as load members, the load members may have other suitable configurations such as an obstruction within the trocar which is overcome by a predetermined force may be used with the present invention. Furthermore, the load mechanism employed by the present invention may be other than a compressive load. Other types of loads that involve torsional forces or tensile forces or other types of forces.

At the proximal end of push rod 36 is locking mechanism 52 for locking onto push rod 36 when sufficient force is applied to the coaxially aligned assembly. Locking mechanism 52 is also biased requiring a minimum force or load equivalent to the combined resistive loads of first load member and second load member in order to overcome the bias and lock onto rod 36. Trocar 32 further includes an implant restraining mechanism such as a leaf spring located within the wall of trocar 32 in the general area indicated by reference arrow 60. The restraining mechanism serves to retain the implant within the trocar once the implant is translated past it in a proximal direction. Device 30 further includes a lever 48 attached at its base to a tubular segment 56. Tubular segment 56 resides coaxially about rod 36 and is translatable within a channel 50 along the length of rod 36 from a first, engaged or loaded position, i.e., prior to operative loading of an implant, to a second, unengaged or unloaded position, as shown in FIG. 2. The change in position of the lever is performed manually.

FIG. 3 illustrates a top cross-sectional view of another embodiment of the present invention. Here, actuation/activation device 70 includes a trocar 72 and a handle 74 within which the proximal end of trocar 72 is affixed. The load assembly within the trocar lumen includes a push rod 76 and a load member or spring 80. The proximal end of push rod 76 has an annular receptacle 78 with a center pin 82 extending proximally into the lumen defined by spring 80. Radially extending around the edge of receptacle 78 is a lip 88. Extending radially inward from the wall of handle 74 is an annular locking shoulder or plate 86. As push rod 76 is translated proximally, the inner edge of locking shoulder 86 engages against lip 88 causing the receptacle walls to flex inward. When lip 88 pass through the passage defined by shoulder 86, lip 88 catches on shoulder 86 thereby preventing distal translation of push rod 76. At the distal end of trocar 72 is an implant retaining member 84 in the form of a leaf spring which prevents a loaded implant from falling out of the trocar 72. The functional aspects and properties of device 70 are similar to those of device 30 of FIG. 2, the latter of which is described in detail below.

The trocars of the present invention may have a total length in the range from about 1 cm to about 20 cm, and more typically in the range about 2 cm to about 10 cm; and an internal diameter in the range from about 0.5 mm to about 6 mm, and more typically in the range about 1 mm to about 4 mm; however, such dimensions may vary depending on the size of the implant device.

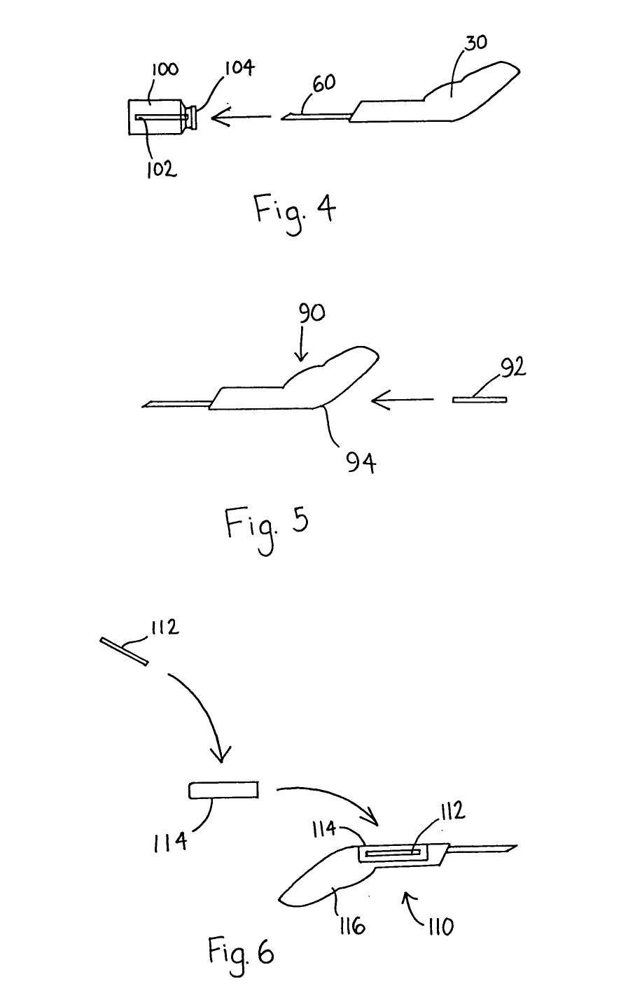

Prior to use, the implant will typically be contained in a channel of a sterile vial, such as vial 100 in FIG. 4, and positioned within the channel 102 such that the valve or actuation/activation mechanism (in this case, the orifice end) of the implant is at the top end or mouth 104 of vial 100 (or optionally it may alternatively be at the bottom end of the vial). Holding handle 34 of device 30, the user inserts the distal end of trocar 56 into channel 102 of vial 100 such that the exposed end of the implant (not shown) is inserted into distal end of the trocar 56 and engages light spring 42. (Of course, the implant may be handled directly by the user and manually inserted into the trocar; however, such may not be preferred as it may comprise the sterility of the implant.) The user will continue to push forward on the trocar handle with sufficient compressive force such that the implant is caused to translate proximally within the trocar lumen, compressing against light spring 42 which in turn compresses against load spacer 40 which in turn compresses against heavy spring 38 which in turn compresses against rod 36 until sufficient force is placed against locking mechanism 52 causing it to lock.

The trocar is designed such that the total resistive load within its lumen, i.e., the compressive load required to actuate locking mechanism 52, is greater than the compressive load required to actuate/activate the implant, in this case, to push the valve in. This is to ensure that the implant cannot be completely loaded until it is activated in order to avoid implanting an inactivated implant. An inactivated implant would provide no beneficial effect to the patient. Thus, when sufficient compressive force, e.g., 2.5 lbf, is applied to the implant and in turn on the loaded assembly within the trocar, the implant is actuated. An additional compressive force, e.g., 5 lbf, is then required to overcome the total resistive force, e.g., 7.5 lbf, of the load members in order to actuate locking mechanism 52. The locking mechanism may be designed to provide tactile or auditory feedback (such as a “click”) to indicate to the user that the locking mechanism has been activated. The linear translation required by the implant in order to provide such additional compressive force causes the implant to translate past the implant restraining member 60, thereby preventing the implant from translating distally back out of the trocar until positive action is taken by the user, i.e., until lever 48 is actuated. Requiring an additional force to load the implant ensures that the implant is activated and properly loaded. If an insufficient amount of additional force is applied, wherein the retaining mechanism has not been engaged, the resulting counter force of the load members will eject the implant out of the trocar. The implant is now properly loaded within the trocar and is activated so that, upon implant, delivery of the beneficial agent will commence.

To implant the device, the physician or user penetrates the tip of the trocar into the patient a selected or predetermined distance, which may be indicated by a marker on the outer wall of the trocar. When this distance is achieved, the user pulls back on lever 48, retracting the trocar. The retracting action removes the trocar from the penetration site leaving the implant behind.

While particular embodiments of the actuation/activation devices of the present invention have thus far been described, it should be understood that other embodiments are possible. For example, the devices may be configured to provide for the backend-loading or lateral-loading of an implant therein. FIG. 5 illustrates a device 90 configured to receive an implant device 92 into its proximal or backend 94. FIG. 6 illustrates another device 110 configured to receive an implant device 112 into the side, top or bottom of handle 116. Implant 112 may be contained in a cartridge 114 which is then loaded into the delivery device 110. In another example, the implanter may have a trocar that does not retract but instead has a push-rod that moves forward and pushes the implant out of the end of the trocar.

Kits

Also provided by the subject invention are kits which include at least one actuation/activation device. The kits may further include at least one implant device contained in a sterile package as well as contain a loading receptacle which is itself loaded onto or into the delivery device. The kits may further include instructions for using the actuation/activation devices and for loading the implants within the delivery devices. The instructions may be printed on a substrate, such as paper or plastic, etc. As such, the instructions may be present in the kits as a package insert, or on the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

It is evident from the above description and discussion that the above described invention provides devices and procedures by which a device requiring activation or actuation to perform some function, e.g., deliver a beneficial agent, can be more safely and easily activated or actuated. The above described invention provides a number of advantages, including reducing the risk of the device being incorrectly activated and the risk that the user will forget to activate the device. Additionally, the present invention reduces the number of handling steps necessary to prepare, e.g., load, a device for implantation and/or activation, thereby decreasing the risk of comprising the sterility of the device. As such, the subject invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the present invention.

### Claims

Though the invention has been described in reference to certain examples, optionally incorporating various features, the invention is not to be limited to the embodiments described. It is to be understood that the breadth of the present invention is to be limited only by the literal or equitable scope of the following claims. That being said, we claim:

What is claimed:

1. An apparatus for activating a function of a device, comprising:
an elongated tubular member having a lumen extending from a proximal end to a distal end, wherein said lumen is adapted to translatably receive said device therein;
a retaining mechanism located within said lumen at said distal end for retaining said device within said lumen when said device is translated proximally within said lumen;
a resistive load means positioned within said lumen and adapted to contact said device received within said lumen, said resistive load means having a first resistive load level and a second resistive load level wherein said second resistive load level is greater than said first resistive load level;
a rod positioned within said lumen proximal to said resistive load means, said rod being translatable within said lumen; and
a locking mechanism for locking onto said rod;
wherein a force applied to said device is in turn applied to said resistive load means wherein, when said applied force is sufficient to overcome said first resistive load level, said device function is activated and further, when said applied force is sufficient to overcome said second resistive load level, said locking mechanism locks onto said rod.

2. The apparatus of claim 1 wherein said resistive load means comprises a spring.

3. The apparatus of claim 1 wherein said second resistive load is greater than said first resistive load.

4. The apparatus of claim 1 wherein the resistive load means comprises at least two resistive load members, and wherein said load members comprise a first load member and a second load member.

5. The apparatus of claim 4 wherein the second load member has a resistive load that is about 2 times to about 10 times that of the first load member.

6. The apparatus of claim 4 wherein the first load member has a resistive load of about 0.5 to 3 lbft, and the second load member has a resistive load of about 1 to 30 lbft.

7. The apparatus of claim 1 wherein said activating of said device is achieved by pushing, pulling, or turning a portion of the device.

8. The apparatus of claim 1 further comprising, at the distal end, an implant-retaining member.

9. The apparatus of claim 8 wherein the implant retaining member comprises a leaf-spring.

10. A kit comprising the apparatus of claim 1 and a drug delivery device the function of which is designed for activation by use of the apparatus.
